(19) United States
(12) Patent Application Publication
(10) Pub. No.: US 2013/0053720 A1
(43) Pub. Date: Feb. 28, 2013

(54) INFORMATION PROCESSOR AND PROCESSING METHOD, PROGRAM AND RECORDING MEDIA

(71) Applicants: Tatsumi Sakaguchi, Kanagawa (JP); Junichiro Enoki, Tokyo (JP); Haruhiko Soma, Tokyo (JP); Takuro Yamamoto, Kanagawa (JP); Mitsuhiro Nakamura, Kanagawa (JP); Tomiji Tanaka, Saitama (JP)

(72) Inventors: Tatsumi Sakaguchi, Kanagawa (JP); Junichiro Enoki, Tokyo (JP); Haruhiko Soma, Tokyo (JP); Takuro Yamamoto, Kanagawa (JP); Mitsuhiro Nakamura, Kanagawa (JP); Tomiji Tanaka, Saitama (JP)

(73) Assignee: SONY CORPORATION, Tokyo (JP)

(21) Appl. No.: 13/564,061

(22) Filed: Aug. 1, 2012

(30) Foreign Application Priority Data
Aug. 22, 2011 (JP) 2011-180088

(51) Int. Cl. A61B 5/0484

(52) U.S. Cl. 600/544

(57) ABSTRACT

An information processor includes a brain wave sensor adapted to output a brain wave signal by measuring brain waves of a subject, an external sensor adapted to sense the surrounding environment of the subject, an environmental change determination section adapted to determine, based on a sensor signal output from the external sensor, whether a stimulus resulting from a change in the surrounding environment of the subject beyond a steady state has been applied to the subject, an event-related potential detection section adapted to detect an event-related potential for the stimulus in the brain wave signal if it is determined that the stimulus has been applied to the subject, and a calculation section adapted to calculate, based on a feature quantity obtained from the detected event-related potential, a value representing the focus level of the subject on a target.

### BACKGROUND

The present technology relates to an information processor and processing method, program and recording media, and more particularly, to an information processor and processing method, program and recording media for identifying the focus level on a specific target more accurately.

It has been common to measure the focus level based on a biosignal.

For example, a technique has been proposed to determine the occurrence of a saccade based on the measured ocular movement, thus detecting the end of the saccade and extracting chronological brain wave signal data within a predetermined period of time from the end of the saccade (refer, for example, to Japanese Patent Laid-Open No. 2010-057710). The technique disclosed in Japanese Patent Laid-Open No. 2010-057710 detects a peak-to-peak value representing the difference between the maximum and minimum brain wave signal levels from chronological data obtained by adding up and averaging a plurality of pieces of chronological data, calculating the level of caution and focus of a driver by multiplying the peak-to-peak value by the number of occurrences of a saccade.

Further, a lecture support system has been proposed which can identify the condition of a student attending a lecture by detecting his or her biological information so as to effectively offer a review session or extra lecture if the student's condition is not favorable (refer, for example, to Japanese Patent Laid-Open No. 2006-293038).

### SUMMARY

However, biological information has not been analyzed in consideration of a stimulus such as light or sound occurring around the subject in related art.

Further, it has been difficult to detect, in related art, whether a subject is focused on a specific target.

For example, the technique disclosed in Japanese Patent Laid-Open No. 2010-057710 determines whether one is highly focused using eye fixation related potential, i.e., a kind of brain wave among biological information. However, it is difficult to determine whether one is focused on a specific piece of content using the technique disclosed in Japanese Patent Laid-Open No. 2010-057710.

Further, although it has been possible to primarily detect whether one is focused in related art, it has been difficult to intentionally detect the focus level at a predetermined timing.

The present technology is disclosed in light of the foregoing, and it is desirable to identify the focus level on a specific target with higher accuracy.

According to an embodiment of the present technology, there is provided an information processor including a brain wave sensor, external sensor, environmental change determination section, event-related potential detection section and calculation section. The brain wave sensor outputs a brain wave signal by measuring brain waves of a subject. The external sensor senses the surrounding environment of the subject. The environmental change determination section determines, based on a sensor signal output from the external sensor, whether a stimulus resulting from a change in the surrounding environment of the subject beyond a steady state has been applied to the subject. The event-related potential detection section detects an event-related potential for the stimulus in the brain wave signal if it is determined that the stimulus has been applied to the subject. The calculation section calculates, based on a feature quantity obtained from the detected event-related potential, a value representing the focus level of the subject on a target.

The information processor can further include a stimulus generation section adapted to generate a stimulus for the target at a preset timing. If the sensor signal, based on which the environmental change determination section determines that a stimulus has been applied to the subject, is not output continuously for a period of time equal to or longer than a predetermined period of time, the stimulus generation section generates the stimulus so that the event-related potential detection section can detect an event-related potential for the generated stimulus.

The information processor can further include a stimulus generation section adapted to generate a stimulus for the target at a preset timing. If a value smaller than a predetermined threshold is obtained by calculation as a value representing the focus level of the subject on a target, the stimulus generation section generates a stimulus so that the event-related potential detection section can detect an event-related potential for the generated stimulus.

The information processor can further include a stimulus generation section adapted to generate a stimulus for the target at a preset timing. A stimulus for the target is generated when an event-related potential is detected which is out of phase with spontaneous activity components of a brain wave signal containing the event-related potential detected earlier by the event-related potential detection section. The calculation section calculates, based on a feature quantity obtained by adding up and averaging a plurality of feature quantities for event-related potentials, a value representing the focus level of the subject on a target.

The information processor can still further include a display section adapted to display a content image serving as the target. A visual stimulus is generated by changing the screen display on the display section at a preset timing.

The information processor can still further include a speaker adapted to produce a content sound serving as the target. An audio stimulus is generated by changing the sound produced from the speaker at a preset timing.

The brain wave sensor can output multi-channel brain wave signals for a plurality of electrodes attached to the head of the subject. The information processor can further include a channel selection section adapted to select a brain wave signal of a predetermined channel for each of the subjects and for the stimuli applied to the subjects from the multi-channel brain wave signals.

According to another embodiment of the present technology, there is provided an information processing method including: using a brain wave sensor to output a brain wave signal by measuring brain waves of a subject; using an external sensor to sense the surrounding environment of the subject; using an environmental change determination section to determine, based on a sensor signal output from the external sensor, whether a stimulus resulting from a change in the surrounding environment of the subject beyond a steady state has been applied to the subject; using an event-related potential detection section to detect an event-related potential for the stimulus in the brain wave signal if it is determined that the stimulus has been applied to the subject; and using a calculation section to calculate, based on a feature quantity obtained from the detected event-related potential, a value representing the focus level of the subject on a target.

According to still another embodiment of the present technology, there is provided a program allowing a computer to serve as an information processor including a brain wave sensor, external sensor, environmental change determination section, event-related potential detection section and calculation section. The brain wave sensor outputs a brain wave signal by measuring brain waves of a subject. The external sensor senses the surrounding environment of the subject. The environmental change determination section determines, based on a sensor signal output from the external sensor, whether a stimulus resulting from a change in the surrounding environment of the subject beyond a steady state has been applied to the subject. The event-related potential detection section detects an event-related potential for the stimulus in the brain wave signal if it is determined that the stimulus has been applied to the subject. The calculation section calculates, based on a feature quantity obtained from the detected event-related potential, a value representing the focus level of the subject on a target.

According to further embodiment of the present technology, there is provided a recording media on which the program is recorded. The program allows a computer to serve as an information processor, the information processor including a brain wave sensor, an external sensor, an environmental change determination section, an event-related potential detection section, and a calculation section. The brain wave sensor outputs a brain wave signal by measuring brain waves of a subject. The external sensor senses the surrounding environment of the subject. The environmental change determination section determines, based on a sensor signal output from the external sensor, whether a stimulus resulting from a change in the surrounding environment of the subject beyond a steady state has been applied to the subject. The event-related potential detection section detects an event-related potential for the stimulus in the brain wave signal if it is determined that the stimulus has been applied to the subject. The calculation section calculates, based on a feature quantity obtained from the detected event-related potential, a value representing the focus level of the subject on a target.

The present technology allows to identify the focus level on a specific target with higher accuracy.

### BRIEF DESCRIPTION OF THE DRAWINGS

FIG. 1 is a block diagram illustrating a configuration example of a focus measurement device using the present technology;

FIG. 2 is a diagram illustrating an example of a stimulus presented;

FIGS. 3A and 3B are diagrams describing a change in brain waves when a stimulus is applied;

FIG. 4 is a flowchart describing an example of a measurement process in active mode;

FIG. 5 is a flowchart describing an example of a measurement process in passive mode;

FIG. 6 is a flowchart describing an example of a measurement process in hybrid mode;

FIG. 7 is a flowchart describing another example of a measurement process in hybrid mode; and

FIG. 8 is a block diagram illustrating a configuration example of a personal computer.

### DETAILED DESCRIPTION OF THE PREFERRED EMBODIMENT

A description will be given below of the preferred embodiment of the technology disclosed in the present specification with reference to the accompanying drawings.

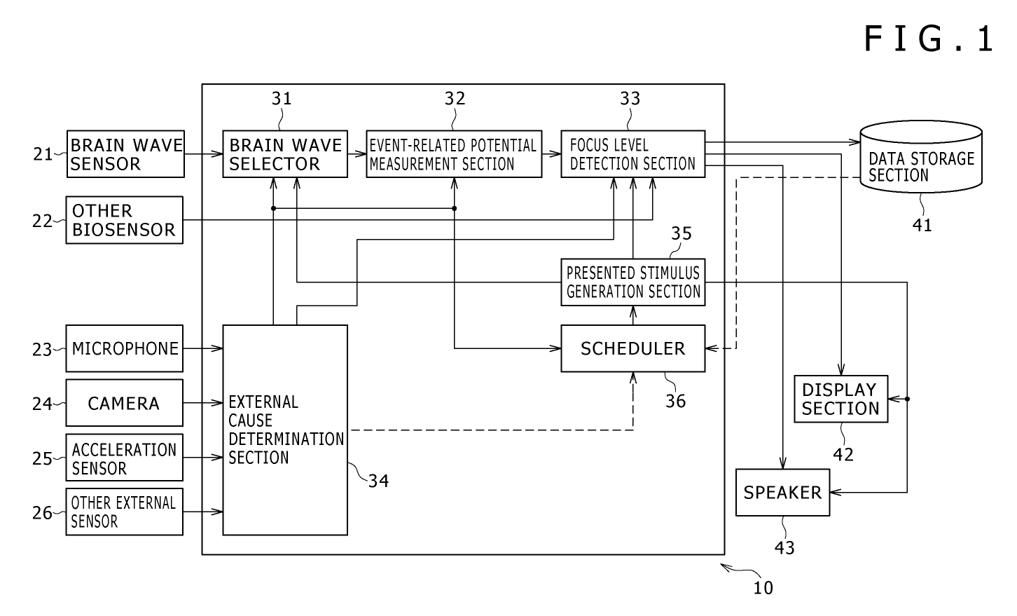

FIG. 1 is a block diagram illustrating a configuration example of a focus measurement device using the present technology. For example, a focus measurement device 10 quantitatively measures the focus level of a subject on a target. The target is something looked at, listened to, and read by the subject, and is, for example, content viewed by the subject. Alternatively, the target may be a lecture attended by the subject. It should be noted that the target, although called as such here, does not necessarily refer to a tangible object.

Further, the person whose focus level is to be measured by the focus measurement device 10 is referred to as the subject here. Therefore, the subject may measure his or her own focus level by using the focus measurement device 10.

A brain wave sensor 21 adapted to measure brain waves of the subject and other biosensor 22 are provided for the focus measurement device 10.

For example, electrodes are attached to the subject so that cerebral activity signals of one or more predetermined areas of the head can be detected. It is preferred that these predetermined areas should be near the top of the head, and that electrodes should be installed at the locations Cz, FZ and Pz according to the International 10-20 System. It should be noted, however, that the locations may be changed from one subject to another to accommodate the individual difference. The brain wave sensor 21 acquires, for example, multi-channel brain wave signals for a plurality of electrodes attached to the head of the subject, supplying the signals to a brain wave selector 31.

It should be noted that the other biosensor 22 includes, for example, a temperature sensor adapted to measure the body temperature of the subject or pulse sensor adapted to measure the pulse of the subject and is provided as necessary. The other biosensor 22 outputs a sensor signal for the measurement result.

Further, a microphone 23, camera 24, acceleration sensor 25 and other external sensor 26 are provided for the focus measurement device 10. The microphone 23 collects sounds surrounding the subject. The camera 24 captures an image surrounding the subject. The acceleration sensor 25 detects the motion of the subject. It should be noted that the other external sensor 26 includes, for example, an illumination sensor or odor sensor and is provided as necessary. The microphone 23, camera 24, acceleration sensor 25 and other external sensor 26 output sensor signals respectively for their measurement results.

Further, the brain wave selector 31, an event-related potential measurement section 32 and focus level detection section 33 are incorporated in the focus measurement device 10.

The brain wave selector 31 selects and outputs, based on the signals output from an external cause determination section 34 and presented stimulus generation section 35, a brain wave signal of a channel to be used for measurement of an event-related potential which will be described later from among the multi-channel brain wave signals output from the brain wave sensor 21.

The event-related potential measurement section 32 measures, in brain waves of the subject, an event-related potential observed in response to a predetermined stimulus. The same section 32 measures the change in potential of the brain wave signal supplied from the brain wave selector 31, thus detecting, for example, a wave referred to as a so-called “P300” waveform.

The focus level detection section 33 calculates the focus level of the subject on the target as a quantitative value based on the measurement result of the event-related potential measurement section 32.

Further, the external cause determination section 34 is incorporated in the focus measurement device 10. The same section 34 identifies the stimulus applied to the subject based on the sensor signals output from the microphone 23, camera 24, acceleration sensor 25 and other external sensor 26. For example, if a sensor signal for a large sound, sharp change in contrast or vigorous motion is input, the external cause determination section 34 outputs a detection signal representing the detection of the sensor signal to the brain wave selector 31 and focus level detection section 33.

Still further, the presented stimulus generation section 35 and a scheduler 36 are incorporated in the focus measurement device 10.

The presented stimulus generation section 35 generates a stimulus to be presented to the subject by controlling a display section 29 and speaker 30 which will be described later, for example, based on a control signal output from the scheduler 36. The stimulus to be presented to the subject is, for example, a change in intervals at which the screen color of the display section 29 changes or a change in sound produced from the speaker.

The scheduler 36 controls the timing at which the presented stimulus generation section 35 generates a stimulus. For example, the scheduler 36 incorporates a timer and controls, based on schedule information registered in advance, the presented stimulus generation section 35 to generate a stimulus such as a change in intervals at which the screen color of the display section 29 changes or a change in sound produced from the speaker at a predetermined timing.

Still further, a data storage section 41, the display section 42 and speaker 43 are provided for the focus measurement device 10.

The data storage section 41 stores, for example, the results output from the focus level detection section 33. The same section 41 stores, for example, data representing the chronological change in focus level for each of the subjects.

The display section 42 is a display that includes, for example, an LCD (liquid crystal display) and displays a content image reproduced by an unshown content reproduction section or other section. Further, the display section 42 changes the screen display under control of the presented stimulus generation section 35 as described above.

The speaker 43 produces a content sound reproduced by an unshown content reproduction section or other section. Further, the speaker 43 changes the produced sound under control of the presented stimulus generation section 35 as described above.

The focus measurement device 10 operates in one of three different modes, namely, active mode, passive mode, and hybrid mode, i.e., a mode that combines active and passive modes.

Active mode is an operation mode designed to quantitatively measure the focus level of a subject on a target by intentionally applying a stimulus to the subject via the target and observing the response to the stimulus. On the other hand, passive mode is an operation mode designed to quantitatively measure the focus level of a subject on a target by using, for example, sound, light or vibration occurring around the subject as a stimulus applied to the subject and observing the response to the stimulus. Hybrid mode is an operation mode designed to operate the focus measurement device 10 in active or passive mode as necessary.

A description will be given here of how the focus level is measured when the focus measurement device 10 operates in active mode.

It is effective to operate the focus measurement device 10 in active mode in order to measure, in a short period of time, the extent to which the subject is focused on viewing content. In this case, the measurer registers schedule information in the scheduler 36. The schedule information includes, for example, the nature of stimulus applied to the subject (e.g., screen color change or sound change) and the time when the stimulus is applied.

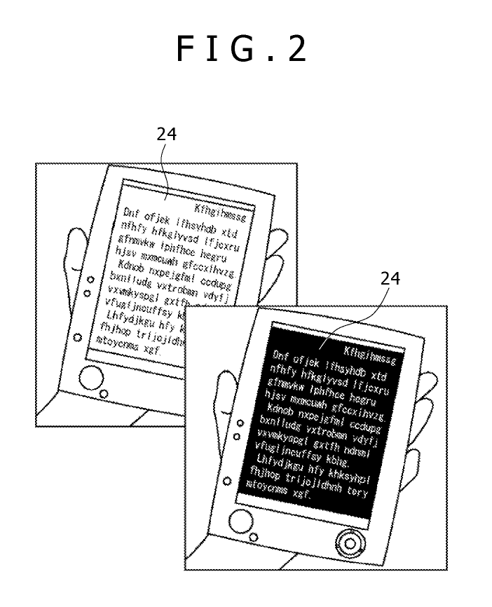

For example, as illustrated in FIG. 2, the background color of the screen of the display section 42 starts to change gently and periodically simultaneously when the viewing of content begins. It should be noted that the background color of the screen undergoes an extreme change in FIG. 2 for reasons of easy understanding. Practically, however, the background color changes in an unannoying manner. Then, the background color of the screen of the display section 42 starts to change more frequently in a predetermined period of time after the viewing of content begins, thus applying a stimulus to the subject.

Alternatively, a small environmental sound starts to be produced from the speaker 43 simultaneously when the viewing of content begins, and this environmental sound is repeated at predetermined intervals. Then, the environmental sound starts to be produced from the speaker 43 more frequently in a predetermined period of time after the viewing of content begins, thus applying a stimulus to the subject.

In addition to the above, other stimuli are also possible such as, firstly, moving the background periodically on the screen of the display section 42 and suddenly changing the movement, and secondly, changing the brightness of the background near the area on which the subject is likely focused, moving the background thereof or changing the color of the background thereof. Still other possible stimuli are suddenly changing the pitch or volume of music produced by the speaker 43 and changing the balance between the left and right speakers in stereo configuration.

It is preferred that a stimulus should be applied to something that undergoes a periodic change in a steady state according to the nature of content and viewing condition so that it breaks away from the periodicity. It is further preferred that the above stimulus should be, for example, applied continuously a plurality of times per measurement so as to ensure higher measurement accuracy. Still further, the intensity of stimulus to be applied may be adjusted, for example, by calibrating the intensity for each user in advance, thus ensuring higher measurement accuracy.

The presented stimulus generation section 35 generates a stimulus based on a control signal output from the scheduler 36 and outputs a signal representing the nature of the generated stimulus to the brain wave selector 31.

The brain wave selector 31 selects, based on the signal supplied from the presented stimulus generation section 35, the channel for the brain wave signal to be supplied to the event-related potential measurement section 32. For example, if the nature of the stimulus to be applied to the subject is to change the screen color, a channel adapted to measure brain waves that are likely to respond to a visual stimulus is selected. On the other hand, if the nature of the stimulus to be applied to the subject is to change the sound, a channel adapted to measure brain waves that are likely to respond to an audio stimulus is selected.

Further, the brain wave selector 31 sets up a frequency filter to allow the event-related potential measurement section 32 to extract a frequency waveform suitable for detection of a P300 waveform in the brain wave signal of the channel.

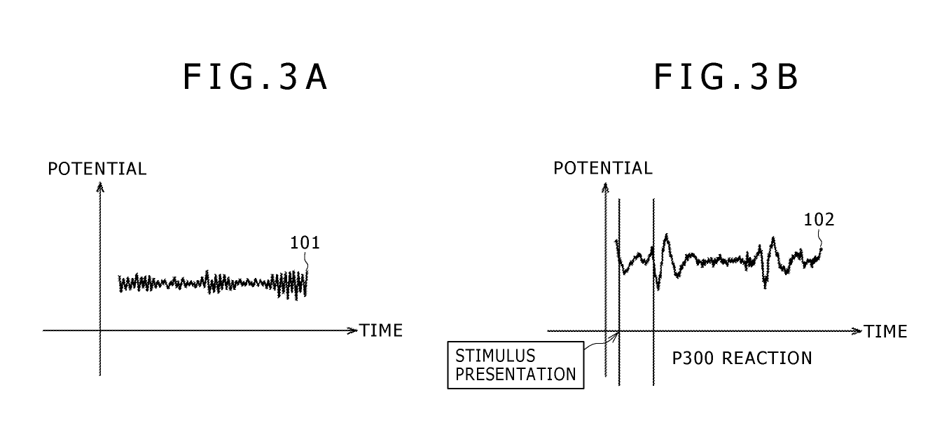

As shown in FIG. 3A, for example, if no stimulus is applied, a brain wave signal output from the brain wave selector 31 has a small amplitude as does a waveform 101. On the other hand, as shown in FIG. 3B, if a stimulus is applied, a brain wave signal output from the brain wave selector 31 has a large amplitude as does a waveform 102.

The event-related potential measurement section 32 analyzes the waveform of a brain wave signal output from the brain wave selector 31, thus detecting a waveform assumed to represent the event-related potential.

A waveform output from the brain wave sensor 21 contains much noise such as myogenic potential and spontaneous activity components. Therefore, a waveform assumed to represent an event-related potential is detected in the following manner.

First, a brain wave signal is recorded as brain wave data for a stimulus for a predetermined time range relative to the time when the stimulus was applied. Then, each time a similar stimulus (e.g., stimulus generated continuously for each measurement) is applied, brain wave data for each of the stimuli is recorded. The plurality of pieces of brain wave data obtained as described above are added up and averaged, thus attenuating spontaneous activity components such as alpha and beta waves and making it easy to detect a waveform assumed to represent an event-related potential.

For example, if the focus level is measured in real time, n pieces of most recent brain wave data are added up and averaged, thus calculating the event-related potential at that time. On the other hand, if the focus level is not measured in real time (as when data is processed in a batch fashion), n/2 pieces of past brain wave data (earlier in time) and n/2 pieces of future brain wave data (later in time) relative to that time are added up and averaged, thus calculating the event-related potential at that time.

The event-related potential measurement section 32 determines, for example, whether a P300 waveform having its peak in about 300 msec after the presentation of a stimulus has been detected in the waveform 102. When it is determined that a P300 waveform has been detected, the same section 32 outputs information including the waveform of an event-related potential (P300 waveform in this case), the time position of that waveform, its frequency and its signal intensity to the focus level detection section 33.

For example, if brain wave signals of a plurality of channels are selected by and output from the brain wave selector 31, the event-related potential measurement section 32 outputs information relating to the signal waveform of the channel from which a waveform closest to a P300 waveform was detected to the focus level detection section 33. Such information includes the time position of the waveform, its frequency and its signal intensity. That is, the event-related potential measurement section 32 outputs a feature quantity relating to the detected event-related potential waveform to the focus level detection section 33.

The focus level detection section 33 calculates, based on the information supplied from the event-related potential measurement section 32, a value representing the focus level of the subject on a target.

Here, the relative intensity of the stimulus with respect to the event-related potential waveform is denoted by vs, the maximum amplitude of the waveform vr, the amount of time from when the stimulus is presented to when the peak of the waveform is reached tp, the start time when the waveform begins to appear ts, the time when the waveform is extracted Δt, and the amplitude at each of times t during that period v(t). Then, a focus level cs of the subject on content in active mode is expressed by Equation (1).

cs=f(vr,vs)+g(tp,vs)+h(v(t),ts,Δt,vs) (1)

It should be noted, however, that each term of Equation (1) is defined by Equations (2), (3) and (4).

$$\begin{matrix}f ( vr , vs ) = α \frac{\vert vr \vert}{vs} & ( 2 ) \\ g ( vr , vs ) = β \frac{t}{vs} & ( 3 ) \\ h ( v ( t ) , ts , Δ t , vs ) = {\int }_{t = ts}^{ts + Δ t} γ \frac{\vert v ( t ) \vert}{vs} d t & ( 4 )\end{matrix}$$

α in Equation (2), β in Equation (3) and γ in Equation (4) may be found by learning in advance using a number of pieces of data. Alternatively, appropriate values may be set for each subject.

The stronger (more obvious) the response to the applied stimulus, the higher the value calculated using Equation (1). For example, if the change in color of the screen on which content, i.e., the target, appears, is applied as a stimulus, it is probable that the subject may be focused on the content when he or she responds strongly to the stimulus. In contrast, if the subject responds weakly to the stimulus, it is probable that he or she may not be focused on the content.

It should be noted that if the other biosensor 22 is used in addition to the brain wave sensor 21, a term should be generated for converting the intensity of reaction to the stimulus into a numerical value for each of the sensor signals output from the other biosensor 22 and introduce this term into Equation (1) so as to calculate the focus level cs of the subject on the content.

The focus level detection section 33 calculates the focus level as described above. Then, the calculated focus level is, for example, brought into correlation with information supplied from the event-related potential measurement section 32 and stored in the data storage section 41 as chronological data for each subject.

Further, the data stored in the data storage section 41 may be, for example, analyzed so as to feed back the channel from which a waveform assumed to represent the event-related potential is detected, frequency of the waveform, amount of time until the waveform appears and other information to the brain wave selector 31 or event-related potential measurement section 32.

This makes it possible, for example, to select a different channel and set the frequency filter at different frequencies for each subject, thus allowing detection of a waveform assumed to represent an event-related potential with high accuracy in accordance with biological features of the subject.

A description has been given here of an example in which the focus level is calculated when a stimulus is generated and applied by the presented stimulus generation section 35. However, for example, the nature of stimulus generated by the presented stimulus generation section 35 may be changed so that a plurality of stimuli are applied within a predetermined period of time. For example, five stimuli caused by the change in screen color and five stimuli caused by the change in sound may be applied in a time zone from 30 minutes after the start of the viewing of content to 40 minutes later. Then, the average of the focus levels obtained for the stimuli may be calculated as the focus level of the subject on the content in that time zone.

Further, the event-related potential measurement section 32 adds up and averages a plurality of pieces of brain wave data as described above, thus attenuating spontaneous activity components such as alpha and beta waves and making it easy to detect a waveform assumed to represent an event-related potential. Therefore, each time the same section 32 receives a brain wave signal, the phases of the spontaneous activity components contained in the brain wave signal should be identified. Then, the identified phases of the spontaneous activity components should be fed back to the scheduler 36 so that the scheduler 36 generates a control signal in such a manner as to bring the spontaneous activity components out of phase when a next stimulus is generated.

That is, the scheduler 36 should intentionally fine-tune the times at which stimuli are generated so that the phases of the spontaneous activity components contained in the waveform assumed to represent an event-related potential in response to the stimuli applied earlier are different from the phases of those contained in the waveform assumed to represent an event-related potential in response to the stimuli applied later. This attenuates the spontaneous activity components by adding up and averaging a smaller number of pieces of brain wave data, thus ensuring, for example, high measurement accuracy in a short period of time.

The focus level with the focus measurement device 10 operating in active mode is measured as described above.

A description will be given next of how the focus level is measured when the focus measurement device 10 operates in passive mode.

In order to measure the focus level of the subject without interfering with the focusing of the subject viewing content to the extent possible, for example, it is effective to operate the focus measurement device 10 in passive mode.

The stimulus applied to the subject in passive mode is a change in the surrounding environment of the subject beyond a steady state.

For example, if a large sound is produced outside when the subject is viewing content, the external cause determination section 34 identifies, based on a sensor signal output from the microphone 23, that a stimulus has been applied to the subject. For example, if the amplitude of the sensor signal output from the microphone 23 exceeds the threshold, it is identified that a stimulus has been applied to the subject.

It should be noted that a specific example of a stimulus applied in passive mode is a change in intervals of a sound at regular intervals caused by the rail joints of an electric train when the viewing of content in such an electric train is considered. Other possible examples are a change in surrounding illuminance at the entrance and exit of a tunnel and a change in acceleration resulting from sudden start and stop.

The external cause determination section 34 outputs a detection signal including the nature of stimulus to the brain wave selector 31 and focus level detection section 33.

It should be noted that if, for example, an excessively large sound is produced outside, it is probable that the subject may have difficulty being focused on content because of a stimulus caused by the sound. If a stimulus is applied which obviously interferes with the focusing of the subject as described above, it is no longer meaningful to measure the focus level. Therefore, the process adapted to measure the focus level is reset.

The brain wave selector 31 selects, based on the signal supplied from the external cause determination section 34, the channel for the brain wave signal to be supplied to the event-related potential measurement section 32. For example, if a sound-related stimulus is applied to the subject, a channel adapted to measure brain waves that are likely to respond to an audio stimulus is selected.

Further, the brain wave selector 31 sets up a frequency filter to allow the event-related potential measurement section 32 to extract a frequency waveform suitable for detection of a P300 waveform in the brain wave signal of the channel.

The event-related potential measurement section 32 analyzes the waveform of a brain wave signal output from the brain wave selector 31, thus detecting a waveform assumed to represent an event-related potential. At this time, the plurality of pieces of brain wave data are added up and averaged, thus attenuating spontaneous activity components as described above.

Unlike in active mode, the event-related potential measurement section 32 determines, for example, whether a P300 waveform having its peak in about 300 msec after the presentation of a stimulus has been detected. When it is determined that a P300 waveform has been detected, the same section 32 outputs information including the waveform of an event-related potential (P300 waveform in this case), the time position of that waveform, its frequency and its signal intensity to the focus level detection section 33.

For example, if brain wave signals of a plurality of channels are output from the brain wave selector 31, the event-related potential measurement section 32 outputs information relating to the signal waveform of the channel from which a waveform closest to a P300 waveform was detected to the focus level detection section 33. Such information includes the time position of the waveform, its frequency and its signal intensity.

The focus level detection section 33 calculates, based on the information supplied from the event-related potential measurement section 32, a value representing the focus level of the subject on a target. It should be noted that a value representing the focus level of the subject is calculated in passive mode by using an equation different from that used in active mode.

Here, the relative intensity of the stimulus with respect to the event-related potential waveform is denoted by vs, the maximum amplitude of the waveform vr, the amount of time from when the stimulus is presented to when the peak of the waveform is reached tp, the start time when the waveform begins to appear ts, the time when the waveform is extracted Δt, and the amplitude at each of times t during that period v(t). Then, the focus level cs of the subject on content in passive mode is expressed by Equation (5).

$$cs = \frac{1}{f ( vr , vs ) + g ( tp , vs ) + h ( v ( t ) , ts , Δ t , vs )} \qquad ( 5 )$$

It should be noted, however, that each term of Equation (5) is defined by Equations (6), (7) and (8).

$$\begin{matrix}f ( vr , vs ) = α \frac{\vert vr \vert}{vs} & ( 6 ) \\ g ( vr , vs ) = β \frac{t}{vs} & ( 7 ) \\ h ( v ( t ) , ts , Δ t , vs ) = {\int }_{t = ts}^{ts + Δ t} γ \frac{\vert v ( t ) \vert}{vs} d t & ( 8 )\end{matrix}$$

α in Equation (6), β in Equation (7) and γ in Equation (8) may be found by learning in advance using a number of pieces of data. Alternatively, appropriate values may be set for each subject.

The stronger (more obvious) the response to the applied stimulus, the lower the value calculated using Equation (5). For example, if a sound heard from a location far away from the screen on which content, i.e., the target, appears, is applied as a stimulus, it is probable that the subject may not be focused on the content when he or she responds strongly to the stimulus. In contrast, if the subject responds weakly to the stimulus, it is probable that he or she may be focused on the content.

The focus level detection section 33 calculates the focus level as described above. Then, the calculated focus level is, for example, brought into correlation with information supplied from the event-related potential measurement section 32 and stored in the data storage section 41 as chronological data for each subject.

Further, the data stored in the data storage section 41 may be, for example, analyzed so as to feed back the channel from which a waveform assumed to represent an event-related potential is detected, frequency of the waveform, amount of time until the waveform appears and other information to the brain wave selector 31 or event-related potential measurement section 32.

This makes it possible, for example, to detect a waveform assumed to represent an event-related potential for each user with high accuracy.

Alternatively, for example, the average of the focus levels obtained for a plurality of stimuli applied in a time zone from 30 minutes after the start of the viewing of content to 60 minutes later may be calculated as the focus level of the subject on the content in that time zone.

The focus level with the focus measurement device 10 operating in passive mode is measured as described above.

A description will be given next of how the focus level is measured when the focus measurement device 10 operates in hybrid mode.

In active mode, for example, it is extremely difficult to select the nature of a stimulus. The reason for this is that if a stimulus is applied that may induce a strong response, the stimulus in itself hinders the viewing of content. On the other hand, such a problem is unlikely in passive mode. However, it is difficult to measure the focus level unless a change that can be considered a stimulus occurs.

Therefore, a scheme is, for example, considered which measures the focus level in passive mode and switches to active mode if no stimulus is generated within a certain period of time.

In this case, a timer or other device is incorporated in the external cause determination section 34 so that if it is not identified within a predetermined amount of time that a stimulus has been applied to the subject, the external cause determination section 34 outputs a signal to that effect to the scheduler 36. As a result, it is only necessary for the scheduler 36 to automatically generate schedule information so that the focus level is measured in active mode as described above.

In active mode, on the other hand, the focusing on content may be hindered instead depending on how a stimulus is applied.

Therefore, a scheme is, for example, considered which measures the focus level in active mode for purposes of verification if it is determined as a result of measurement of the focus level in passive mode that the focus level on the content is low.

In this case, for example, the scheduler 36 verifies the focus level of the subject on the content by reading the data stored in the data storage section 41. Then, it is only necessary for the scheduler 36 to automatically generate schedule information so that the focus level is measured in active mode as described above.

The focus level with the focus measurement device 10 operating in hybrid mode is measured as described above.

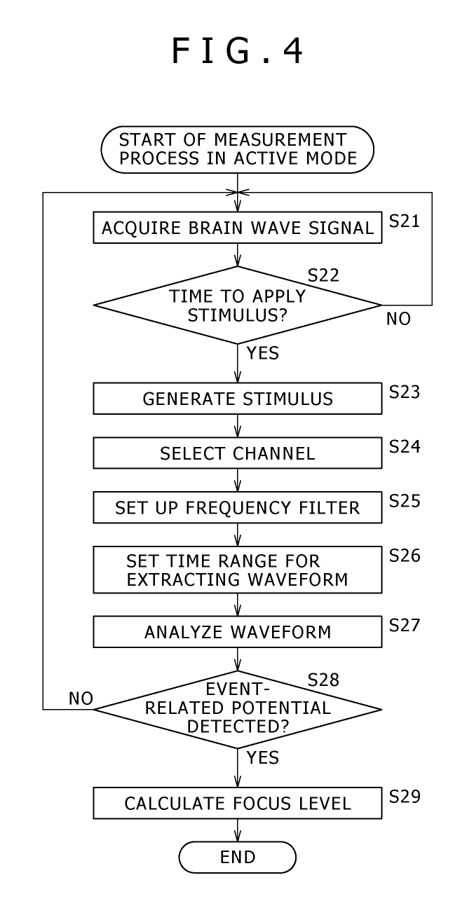

A description will be given next of an example of a measurement process in active mode, i.e., a process adapted to measure the focus level when the focus measurement device 10 operates in active mode, with reference to the flowchart shown in FIG. 4.

This process is performed, for example, to measure the focus level of the subject on the content which he or she is viewing. We assume that a content image is now displayed on the display section 42, and that a content sound is now produced from the speaker 43.

In step S21, the brain wave sensor 21 acquires a brain wave signal. The same sensor 21 acquires, for example, multi-channel brain wave signals for a plurality of electrodes attached to the head of the subject.

In step S22, the scheduler 36 determines whether it is time to apply a stimulus. If it is determined in step S22 that it is not yet time to apply a stimulus, the process returns to step S21. When it is determined in step S22 that it is time to apply a stimulus, the process proceeds to step S23.

In step S23, the scheduler 36 controls the presented stimulus generation section 35 to generate a stimulus. At this time, a stimulus such as a change in intervals at which the screen color of the display section 29 changes or a change in sound produced from the speaker is generated as described above.

In step S24, the brain wave selector 31 selects, based on the signal supplied from the presented stimulus generation section 35, the channel for the brain wave signal to be supplied to the event-related potential measurement section 32. At this time, for example, if the nature of the stimulus to be applied to the subject is to change the screen color, a channel adapted to measure brain waves that are likely to respond to a visual stimulus is selected. On the other hand, if the nature of the stimulus to be applied to the subject is to change the screen sound, a channel adapted to measure brain waves that are likely to respond to an audio stimulus is selected.

In step S25, the brain wave selector 31 sets up a frequency filter to allow the event-related potential measurement section 32 to extract a frequency waveform suitable for detection of a P300 waveform in the brain wave signal of the channel.

In step S26, the event-related potential measurement section 32 sets a predetermined time range for which to record brain wave data for the stimulus applied to the subject as a result of the process in step S23. This time range is set relative to the time when the stimulus was applied.

In step S27, the event-related potential measurement section 32 analyzes the waveform of the brain wave signal of the channel selected by the process in step S24. This waveform is supplied after passing the filter that has been set up by the process in step S25.

At this time, for example, the brain wave signal for the time range set in step S26 is recorded as brain wave data for that stimulus, and then each time a similar stimulus is applied, brain wave data for each of the stimuli will be recorded. For example, if the focus level is measured in real time, n pieces of most recent brain wave data are added up and averaged, thus calculating the event-related potential at that time. On the other hand, if the focus level is not measured in real time (as when data is processed in a batch fashion), n/2 pieces of past brain wave data (earlier in time) and n/2 pieces of future brain wave data (later in time) relative to that time are added up and averaged, thus calculating the event-related potential at that time.

In step S28, the event-related potential measurement section 32 determines whether an event-related potential has been detected as a result of the analysis in step S27. If it is determined in step S28 that an event-related potential has not been detected, the process returns to step S21. When it is determined in step S28 that an event-related potential has been detected, the process proceeds to step S29.

At this time, the event-related potential measurement section 32 determines, for example, whether a P300 waveform having its peak in about 300 msec after the presentation of a stimulus has been detected. When it is determined that a P300 waveform has been detected, the same section 32 outputs information including the waveform of an event-related potential (P300 waveform in this case), the time position of that waveform, its frequency and its signal intensity to the focus level detection section 33.

Further, for example, if brain wave signals of a plurality of channels are output from the brain wave selector 31, the event-related potential measurement section 32 outputs information relating to the signal waveform of the channel from which a waveform closest to a P300 waveform was detected to the focus level detection section 33. Such information includes the time position of the waveform, its frequency and its signal intensity.

In step S29, the focus level detection section 33 calculates the focus level of the subject on the content. At this time, the focus level cs is calculated, for example, by using Equation (1) described above.

The measurement process in active mode is performed as described above.

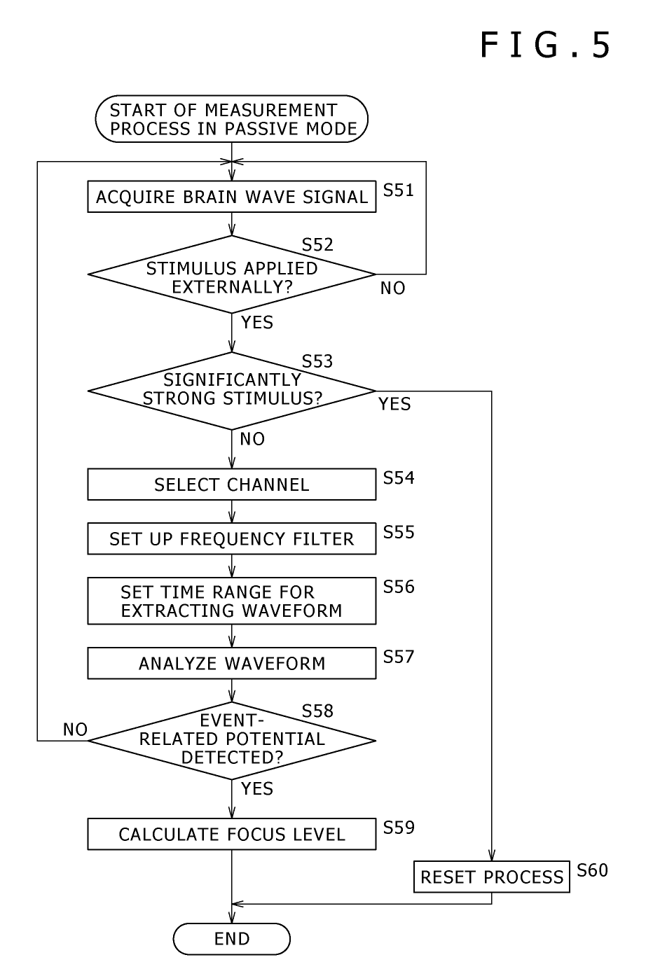

A description will be given next of an example of a measurement process in passive mode, i.e., a process adapted to measure the focus level when the focus measurement device 10 operates in passive mode, with reference to the flowchart shown in FIG. 5.

This process is performed, for example, to measure the focus level of the subject on the content which he or she is viewing. We assume that a content image is now displayed on the display section 42, and that a content sound is now produced from the speaker 43.

In step S51, the brain wave sensor 21 acquires a brain wave signal. The same sensor 21 acquires, for example, multi-channel brain wave signals for a plurality of electrodes attached to the head of the subject.

In step S52, the external cause determination section 34 determines based, for example, on a sensor signal output from the microphone 23, whether a stimulus has been applied to the subject. For example, if the amplitude of the sensor signal output from the microphone 23 exceeds the threshold, it is determined that a stimulus has been applied to the subject.

If it is determined in step S52 that no stimulus has been applied to the subject, the process returns to step S51. When it is determined in step S52 that a stimulus has been applied to the subject, the process proceeds to step S53.

In step S53, it is determined whether the stimulus, determined to have been applied in step S52, was significantly strong. If, for example, an excessively large sound is produced outside, it is probable that the subject may have difficulty being focused because of a stimulus caused by the sound. Whether such a significantly strong stimulus was produced is determined in step S53. For example, if the amplitude of the sensor signal output from the microphone 23 exceeds another threshold (which is greater than that in step S52), it is determined that the stimulus, determined to have been applied in step S52, was significantly strong.

When it is determined in step S53 that the stimulus, determined to have been applied in step S52, was significantly strong, the process proceeds to step S60 where the reset process is performed. For example, if a stimulus was applied which obviously interfered with the focusing of the subject as described above, it is no longer meaningful to measure the focus level. Therefore, the process adapted to measure the focus level is reset.

On the other hand, if it is determined in step S53 that the stimulus, determined to have been applied in step S52, was not significantly strong, the process proceeds to step S54.

In step S54, the brain wave selector 31 selects, based on the signal supplied from the external cause determination section 34, the channel for the brain wave signal to be supplied to the event-related potential measurement section 32. At this time, for example, if a sound-related stimulus was applied to the subject, a channel adapted to measure brain waves that are likely to respond to an audio stimulus is selected.

In step S55, the brain wave selector 31 sets up a frequency filter to allow the event-related potential measurement section 32 to extract a frequency waveform suitable for detection of a P300 waveform in the brain wave signal of the channel.

In step S56, the event-related potential measurement section 32 sets a predetermined time range for which to record brain wave data for the stimulus applied to the subject as a result of the process in step S53. This time range is set relative to the time when the stimulus was determined to have been applied.

In step S57, the event-related potential measurement section 32 analyzes the waveform of the brain wave signal of the channel selected by the process in step S54. This waveform is supplied after passing the filter that has been set up by the process in step S55.

At this time, for example, the brain wave signal for the time range set in step S56 is recorded as brain wave data for that stimulus, and then each time a similar stimulus is applied, brain wave data for each of the stimuli will be recorded. For example, if the focus level is measured in real time, n pieces of most recent brain wave data are added up and averaged, thus calculating the event-related potential at that time. On the other hand, if the focus level is not measured in real time (as when data is processed in a batch fashion), n/2 pieces of past brain wave data (earlier in time) and n/2 pieces of future brain wave data (later in time) relative to that time are added up and averaged, thus calculating the event-related potential at that time.

In step S58, the event-related potential measurement section 32 determines whether an event-related potential has been detected as a result of the analysis in step S57. If it is determined in step S58 that an event-related potential has not been detected, the process returns to step S51. When it is determined in step S58 that an event-related potential has been detected, the process proceeds to step S59.

At this time, the event-related potential measurement section 32 determines, for example, whether a P300 waveform having its peak in about 300 msec after the presentation of a stimulus has been detected. When it is determined that a P300 waveform has been detected, the same section 32 outputs information including the waveform of an event-related potential (P300 waveform in this case), the time position of that waveform, its frequency and its signal intensity to the focus level detection section 33.

Further, for example, if brain wave signals of a plurality of channels are output from the brain wave selector 31, the event-related potential measurement section 32 outputs information relating to the signal waveform of the channel from which a waveform closest to a P300 waveform was detected to the focus level detection section 33. Such information includes the time position of the waveform, its frequency and its signal intensity.

In step S59, the focus level detection section 33 calculates the focus level of the subject on the content. At this time, the focus level cs is calculated, for example, by using Equation (5) described above.

The measurement process in passive mode is performed as described above.

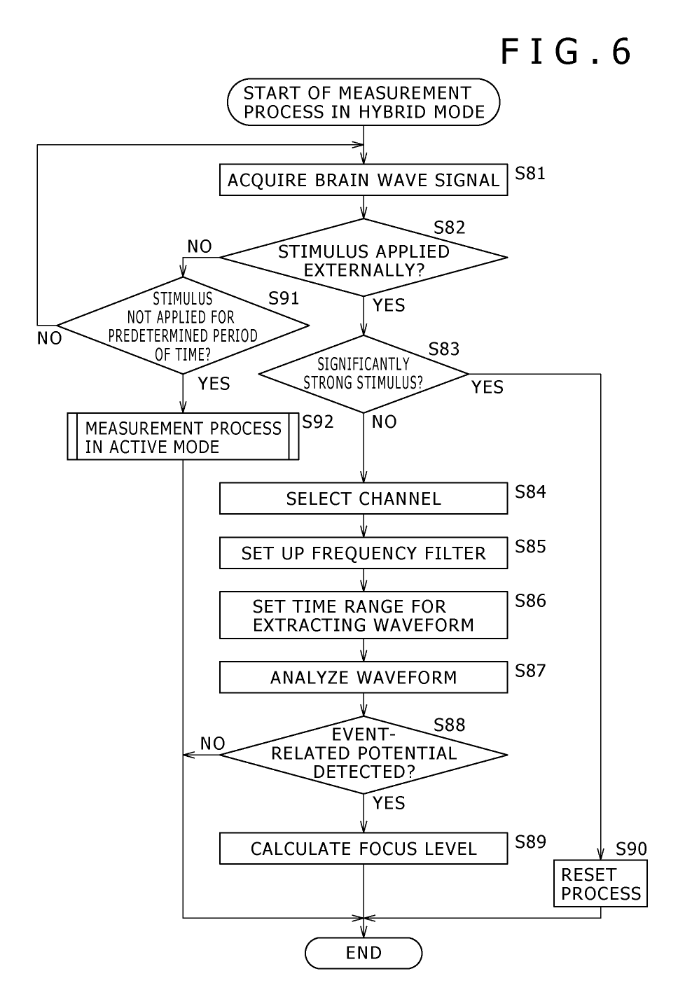

A description will be given next of an example of a measurement process in hybrid mode, i.e., a process adapted to measure the focus level when the focus measurement device 10 operates in hybrid mode, with reference to the flowchart shown in FIG. 6.

This process is performed, for example, to measure the focus level of the subject on the content which he or she is viewing. We assume that a content image is now displayed on the display section 42, and that a content sound is now produced from the speaker 43.

In step S81, the brain wave sensor 21 acquires a brain wave signal. The same sensor 21 acquires, for example, multi-channel brain wave signals for a plurality of electrodes attached to the head of the subject.

In step S82, the external cause determination section 34 determines based, for example, on a sensor signal output from the microphone 23, whether a stimulus has been applied to the subject. For example, if the amplitude of the sensor signal output from the microphone 23 exceeds the threshold, it is determined that a stimulus has been applied to the subject.

If it is determined in step S82 that no stimulus has been applied to the subject, the process proceeds to step S91. When it is determined in step S82 that a stimulus has been applied to the subject, the process proceeds to step S83.

The processes from steps S83 to S90 in FIG. 6 are respectively the same as those from steps S53 to S60 in FIG. 5. Therefore, a detailed description thereof is omitted.

In step S91, it is determined whether no stimulus has been applied to the subject for a predetermined period of time. When it is determined in step S91 that the period of time for which no stimulus has been applied to the subject does not exceed the predetermined period of time, the process returns to step S81. On the other hand, if it is determined in step S91 that no stimulus has been applied to the subject for the predetermined period of time, that is, if the sensor signal, based on which it is determined that a stimulus has been applied to the subject, has not been output continuously for the predetermined period of time, the process proceeds to step S92.

In step S92, the measurement process in active mode described above with reference to FIG. 4 is performed.

The measurement process in hybrid mode is performed as described above.

That is, the measurement process in hybrid mode shown in FIG. 6 measures the focus level in passive mode and switches to active mode if no stimulus is generated within a certain period of time.

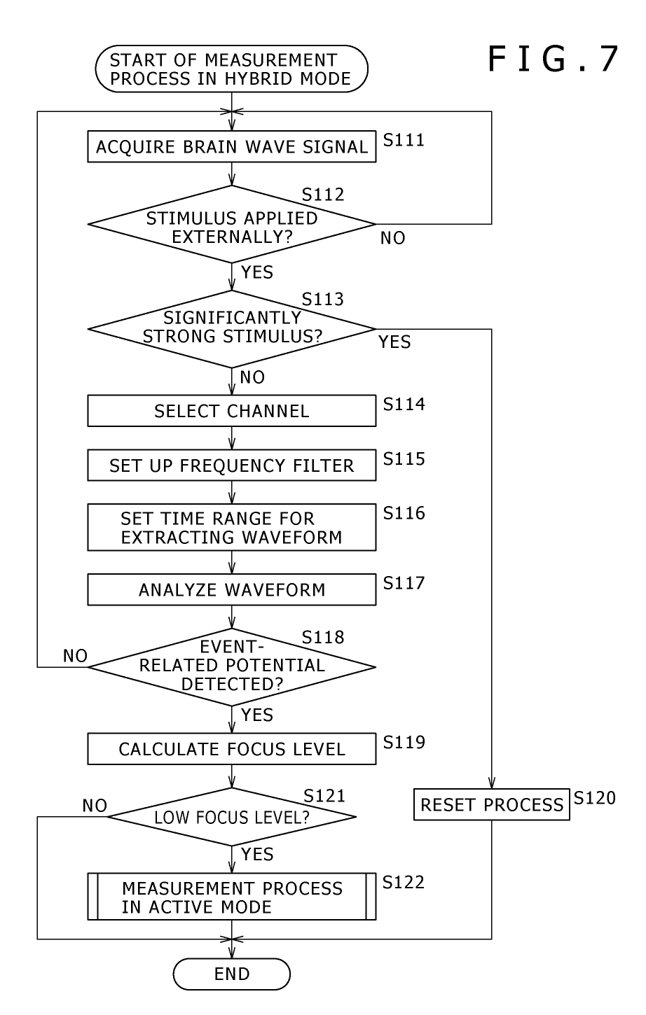

A description will be given next of another example of a measurement process in hybrid mode, i.e., a process adapted to measure the focus level when the focus measurement device 10 operates in hybrid mode, with reference to the flowchart shown in FIG. 7.

This process is performed, for example, to measure the focus level of the subject on the content which he or she is viewing. We assume that a content image is now displayed on the display section 42, and that a content sound is now produced from the speaker 43.

The processes from steps S111 to S120 in FIG. 7 are respectively the same as those from steps S51 to S60 in FIG. 5. Therefore, a detailed description thereof is omitted.

After the process in step S119, the process proceeds to step S121.

In step S121, it is determined whether the focus level calculated in step S119 is low. For example, if the focus level calculated in step S119 is lower than the preset threshold, it is determined in step S121 that the focus level is low.

If it is determined in step S121 that the focus level is low, the process proceeds to step S122.

In step S122, the measurement process in active mode described above with reference to FIG. 4 is performed.

On the other hand, when it is determined in step S121 that the focus level is not low, step S122 is skipped.

The measurement process in hybrid mode is performed as described above.

That is, the measurement process in hybrid mode shown in FIG. 7 measures the focus level in active mode for purposes of verification if it is determined as a result of measurement of the focus level in passive mode that the focus level on the content is low.

It has been difficult in related art to determine, for example, whether one is focused on a particular piece of content. Further, it has been difficult in related art to quantitatively identify the focus level although it has been possible in related art to primarily detect whether one is focused or not. Still further, it has been difficult to intentionally detect the focus level at a predetermined timing.

In contrast, the present technology allows to determine whether one is focused on a specific piece of content. Further, the present technology allows to quantitatively calculate the focus level using Equations 1 and 5 described above. Still further, the present technology allows to intentionally detect the focus level at a predetermined timing by using active mode.

Therefore, the present technology allows, for example, to effectively measure the focus level of a student on a given lecture in a short period of time. Further, the present technology allows, for example, to effectively conduct marketing research on content such as a game or movie in a short period of time.

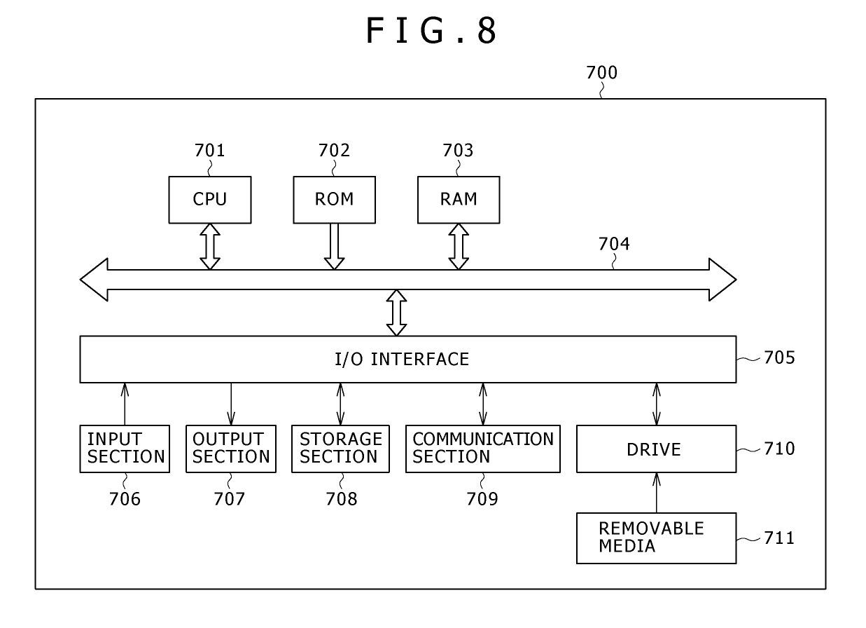

It should be noted that the above series of processes may be performed by hardware or software. If the series of processes are performed by software, the program making up the software is installed from a network or recording media to a computer incorporated in dedicated hardware or a general-purpose personal computer 700 as shown in FIG. 8 capable of performing various functions when installed with various programs.

In FIG. 8, a CPU (Central Processing Unit) 701 performs various processes according to the program stored in a ROM (Read Only Memory) 702 or that loaded from a storage section 708 into a RAM (Random Access Memory) 703. The RAM 703 also stores, as appropriate, data necessary for the CPU 701 to perform various processes.

The CPU 701, ROM 702 and RAM 703 are connected to each other via a bus 704. An I/O (Input/Output) interface 705 is also connected to the bus 704.

An input section 706, output section 707, the storage section 708 and a communication section 709 are connected to the I/O interface 705. The input section 706 includes, for example, a keyboard and mouse. The output section 707 includes, for example, a display and speaker. The display includes, for example, an LCD (Liquid Crystal Display). The storage section 708 includes, for example, a hard disk. The communication section 709 includes, for example, a modem and network interface card such as LAN card. The same section 709 handles communication via networks including the Internet.

A drive 710 is also connected, as necessary, to the I/O interface 705. A removable media 711 such as magnetic disk, optical disk, magneto-optical disk, or semiconductor memory is inserted, as appropriate, into the drive 710. The computer program read from the removable media 711 is installed, as necessary, to the storage section 708.

If the above series of processes are performed by software, the program making up the software is installed from a network such as the Internet or a recording media such as the removable media 711.

It should be noted that this recording media includes those made up of the removable media 711 that are distributed separately from the personal computer 700 to deliver the program to the user such as a magnetic disk (including floppy disk (registered trademark)), optical disk (including CD-ROM (Compact Disk-Read Only Memory) and DVD (Digital Versatile Disk)), magneto-optical disk (MD (Mini Disk) (registered trademark)) and a semiconductor memory. This recording media also includes those that are delivered to the user preinstalled in the personal computer 700 such as the ROM 702 storing the program and the hard disk contained in the storage section 708.

It should be noted that the above series of processes described in the present specification include not only those performed chronologically according to the described sequence but also those that are not necessarily performed chronologically but in parallel or individually.

On the other hand, the embodiments of the present technology are not limited to that described above but may be modified in various ways without departing from the scope of the present technology.

It should be noted that the present technology may have the following configurations.

(1) An information processor including:

a brain wave sensor adapted to output a brain wave signal by measuring brain waves of a subject;

an external sensor adapted to sense the surrounding environment of the subject;

an environmental change determination section adapted to determine, based on a sensor signal output from the external sensor, whether a stimulus resulting from a change in the surrounding environment of the subject beyond a steady state has been applied to the subject;

an event-related potential detection section adapted to detect an event-related potential for the stimulus in the brain wave signal if it is determined that the stimulus has been applied to the subject; and

a calculation section adapted to calculate, based on a feature quantity obtained from the detected event-related potential, a value representing the focus level of the subject on a target.

(2) The information processor of feature (1) further including:

a stimulus generation section adapted to generate a stimulus for the target at a preset timing, in which

if the sensor signal, based on which the environmental change determination section determines that a stimulus has been applied to the subject, is not output continuously for a period of time equal to or longer than a predetermined period of time, the stimulus generation section generates the stimulus so that the event-related potential detection section can detect an event-related potential for the generated stimulus.

(3) The information processor of feature (1) further including:

a stimulus generation section adapted to generate a stimulus for the target at a preset timing, in which

if a value smaller than a predetermined threshold is obtained by calculation as a value representing the focus level of the subject on the target, the stimulus generation section generates the stimulus so that the event-related potential detection section can detect an event-related potential for the generated stimulus.

(4) The information processor of feature (1) including:

a stimulus generation section adapted to generate a stimulus for the target at a preset timing, in which

the stimulus for the target is generated when an event-related potential is detected which is out of phase with spontaneous activity components of a brain wave signal containing the event-related potential detected earlier by the event-related potential detection section, and in which

the calculation section calculates, based on a feature quantity obtained by adding up and averaging a plurality of feature quantities for event-related potentials, a value representing the focus level of the subject on the target.

(5) The information processor of any one of features (1) to (4) further including:

a display section adapted to display a content image serving as the target, in which

a visual stimulus is generated by changing the screen display on the display section at a preset timing.

(6) The information processor of any one of features (1) to (5) further including:

a speaker adapted to produce a content sound serving as the target, in which

an audio stimulus is generated by changing the sound produced from the speaker at a preset timing.

(7) The information processor of any one of features (1) to (7), in which

the brain wave sensor outputs multi-channel brain wave signals for a plurality of electrodes attached to the head of the subject, the information processor further including:

a channel selection section adapted to select a brain wave signal of a predetermined channel for each of the subjects and for the stimuli applied to the subjects from the multi-channel brain wave signals.

(8) An information processing method including:

using a brain wave sensor to output a brain wave signal by measuring brain waves of a subject;

using an external sensor to sense the surrounding environment of the subject;

using an environmental change determination section to determine, based on a sensor signal output from the external sensor, whether a stimulus resulting from a change in the surrounding environment of the subject beyond a steady state has been applied to the subject;

using an event-related potential detection section to detect an event-related potential for the stimulus in the brain wave signal if it is determined that the stimulus has been applied to the subject; and

using a calculation section to calculate, based on a feature quantity obtained from the detected event-related potential, a value representing the focus level of the subject on a target.

(9) A program allowing a computer to serve as an information processor, the information processor including:

a brain wave sensor adapted to output a brain wave signal by measuring brain waves of a subject;

an external sensor adapted to sense the surrounding environment of the subject;

an environmental change determination section adapted to determine, based on a sensor signal output from the external sensor, whether a stimulus resulting from a change in the surrounding environment of the subject beyond a steady state has been applied to the subject;

an event-related potential detection section adapted to detect an event-related potential for the stimulus in the brain wave signal if it is determined that the stimulus has been applied to the subject; and

a calculation section adapted to calculate, based on a feature quantity obtained from the detected event-related potential, a value representing the focus level of the subject on a target.

(10) A recording media on which the program is recorded, the program allowing a computer to serve as an information processor, the information processor including:

a brain wave sensor adapted to output a brain wave signal by measuring brain waves of a subject;

an external sensor adapted to sense the surrounding environment of the subject;

an environmental change determination section adapted to determine, based on a sensor signal output from the external sensor, whether a stimulus resulting from a change in the surrounding environment of the subject beyond a steady state has been applied to the subject;

an event-related potential detection section adapted to detect an event-related potential for the stimulus in the brain wave signal if it is determined that the stimulus has been applied to the subject; and

a calculation section adapted to calculate, based on a feature quantity obtained from the detected event-related potential, a value representing the focus level of the subject on a target.

The present disclosure contains subject matter related to that disclosed in Japanese Priority Patent Application JP 2011-180088 filed in the Japan Patent Office on Aug. 22, 2011, the entire content of which is hereby incorporated by reference.

What is claimed is:

1. An information processor comprising:
a brain wave sensor adapted to output a brain wave signal by measuring brain waves of a subject;
an external sensor adapted to sense the surrounding environment of the subject;
an environmental change determination section adapted to determine, based on a sensor signal output from the external sensor, whether a stimulus resulting from a change in the surrounding environment of the subject beyond a steady state has been applied to the subject;
an event-related potential detection section adapted to detect an event-related potential for the stimulus in the brain wave signal if it is determined that the stimulus has been applied to the subject; and
a calculation section adapted to calculate, based on a feature quantity obtained from the detected event-related potential, a value representing the focus level of the subject on a target.

2. The information processor according to claim 1 further comprising:
a stimulus generation section adapted to generate a stimulus for the target at a preset timing, wherein
if the sensor signal, based on which the environmental change determination section determines that a stimulus has been applied to the subject, is not output continuously for a period of time equal to or longer than a predetermined period of time, the stimulus generation section generates the stimulus so that the event-related potential detection section can detect an event-related potential for the generated stimulus.

3. The information processor according to claim 1 further comprising:
a stimulus generation section adapted to generate a stimulus for the target at a preset timing, wherein
if a value smaller than a predetermined threshold is obtained by calculation as a value representing the focus level of the subject on the target, the stimulus generation section generates the stimulus so that the event-related potential detection section can detect an event-related potential for the generated stimulus.

4. The information processor according to claim 1 comprising:
a stimulus generation section adapted to generate a stimulus for the target at a preset timing, wherein
the stimulus for the target is generated when an event-related potential is detected which is out of phase with spontaneous activity components of a brain wave signal containing the event-related potential detected earlier by the event-related potential detection section, and
the calculation section calculates, based on a feature quantity obtained by adding up and averaging a plurality of feature quantities for event-related potentials, a value representing the focus level of the subject on the target.

5. The information processor according to claim 1 further comprising:
a display section adapted to display a content image serving as the target, wherein
a visual stimulus is generated by changing the screen display on the display section at a preset timing.

6. The information processor according to claim 1 further comprising:
a speaker adapted to produce a content sound serving as the target, wherein
an audio stimulus is generated by changing the sound produced from the speaker at a preset timing.

7. The information processor of claim 1, wherein
the brain wave sensor outputs multi-channel brain wave signals for a plurality of electrodes attached to the head of the subject, the information processor further comprising:
a channel selection section adapted to select a brain wave signal of a predetermined channel for each of the subjects and for the stimuli applied to the subjects from the multi-channel brain wave signals.

8. An information processing method comprising:
using a brain wave sensor to output a brain wave signal by measuring brain waves of a subject;
using an external sensor to sense the surrounding environment of the subject;
using an environmental change determination section to determine, based on a sensor signal output from the external sensor, whether a stimulus resulting from a change in the surrounding environment of the subject beyond a steady state has been applied to the subject;
using an event-related potential detection section to detect an event-related potential for the stimulus in the brain wave signal if it is determined that the stimulus has been applied to the subject; and
using a calculation section to calculate, based on a feature quantity obtained from the detected event-related potential, a value representing the focus level of the subject on a target.

9. A program allowing a computer to serve as an information processor, the information processor comprising:
a brain wave sensor adapted to output a brain wave signal by measuring brain waves of a subject;
an external sensor adapted to sense the surrounding environment of the subject;
an environmental change determination section adapted to determine, based on a sensor signal output from the external sensor, whether a stimulus resulting from a change in the surrounding environment of the subject beyond a steady state has been applied to the subject;
an event-related potential detection section adapted to detect an event-related potential for the stimulus in the brain wave signal if it is determined that the stimulus has been applied to the subject; and
a calculation section adapted to calculate, based on a feature quantity obtained from the detected event-related potential, a value representing the focus level of the subject on a target.

10. A recording media on which the program is recorded, the program allowing a computer to serve as an information processor, the information processor including:
a brain wave sensor adapted to output a brain wave signal by measuring brain waves of a subject;
an external sensor adapted to sense the surrounding environment of the subject;
an environmental change determination section adapted to determine, based on a sensor signal output from the external sensor, whether a stimulus resulting from a change in the surrounding environment of the subject beyond a steady state has been applied to the subject;
an event-related potential detection section adapted to detect an event-related potential for the stimulus in the brain wave signal if it is determined that the stimulus has been applied to the subject; and
a calculation section adapted to calculate, based on a feature quantity obtained from the detected event-related potential, a value representing the focus level of the subject on a target.
